(19) United States
(12) Patent Application Publication
(10) Pub. No.: US 2011/0106095 A1
(43) Pub. Date: May 5, 2011

(54) DRILL GUIDE ASSEMBLY

(75) Inventors: Stanley Cross, Leeds (GB); Marcus Andrew Orton, Boston Spa (GB); Magnus Flett, Wakefield (GB); Gary Moore, Wetherby (GB); Tadgh O'Sullivan, Waterford (IE)

(73) Assignee: DEPUY INTERNATIONAL LTD, Leeds (GB)

(21) Appl. No.: 10/589,063

(22) PCT Filed: Feb. 23, 2005
(86) PCT No.: PCT/GB2005/000734

§ 371 Date: Nov. 9, 2007

(30) Foreign Application Priority Data
Feb. 27, 2004 (GB) 0404347.7

(51) Int. Cl. A61B 17/56

(52) U.S. Cl. 606/96

(57) ABSTRACT

A drill guide assembly for determining the axis for drilling a bore in a generally dome-shaped bone to receive a component of an orthopaedic joint prosthesis is described. The drill guide assembly includes a drill guide sleeve, a carriage and a platform. The drill guide sleeve is mounted in the carriage towards a first end so that the angular orientation of the drill guide sleeve relative to the carriage can be adjusted about at least one axis. The carriage includes at least one threaded angle-adjustment screw which extends between the carriage and the drill guide sleeve by which the angular orientation of the drill guide sleeve can be adjusted. The platform is fastenable to the bone, and includes at least three feet depending from the platform to engage the surface of the bone with the bone extending towards the platform into the space between the feet.

The present invention relates to a drill guide assembly for determining the axis for drilling a bore in a bone to receive a component of an orthopaedic joint prosthesis.

When preparing a bone to receive a component of a joint prosthesis, it can be important to establish the position of an axis which provides a reference against which the location of the prosthesis component is determined for implantation. The axis can conveniently be determined prior to any resection of the bone, relative to the natural bone tissue.

When the bone tissue provides the ball component of a ball and socket joint (for example the humeral component of a shoulder joint or, especially, the femoral component of a hip joint), the axis should be determined relative to its convex bearing surface: the prosthesis component should be implanted in alignment with that axis or at a predetermined orientation relative to the axis. It can be difficult for a surgeon to align a drill guide accurately relative to a bone prior to fixing the drill guide to the bone for use.

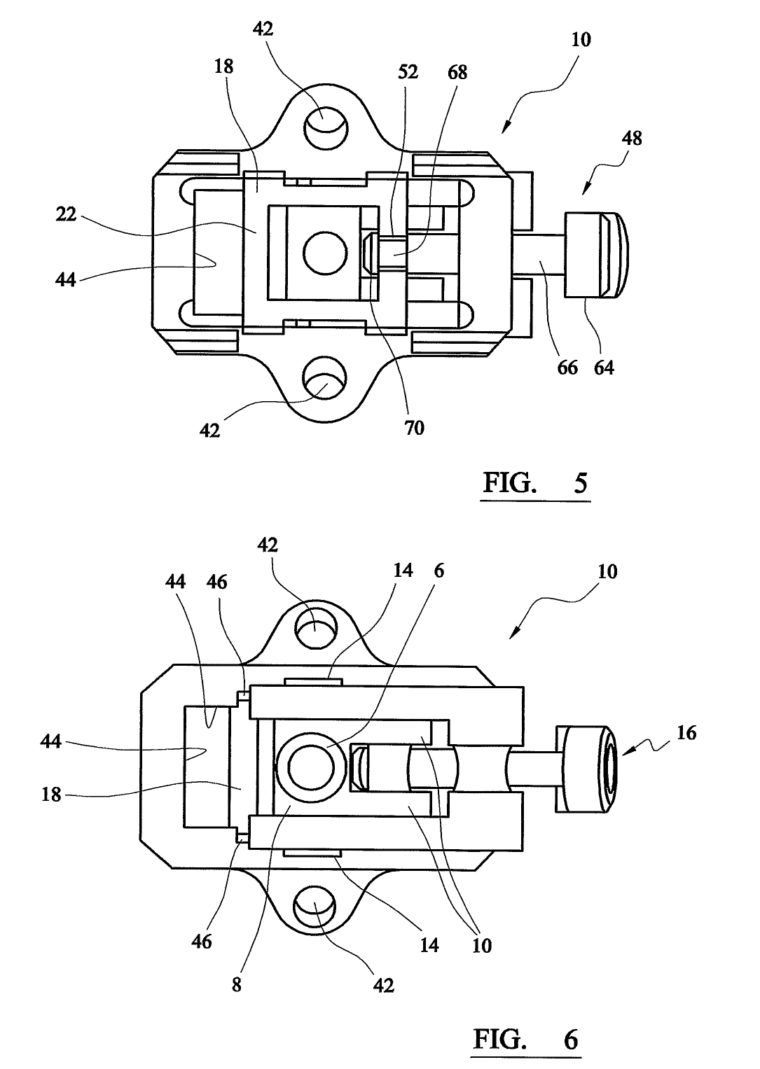

U.S. Pat. No. 6,595,999 discloses a drilling jig which includes a drill guide tube with a rounded head which is supported in a housing. The head is able to pivot within the housing so that the axial orientation of the drill guide tube relative to the housing can be adjusted. The tube can be clamped against adjustment. The clamp comprises upper and lower housing parts which can be drawn together so as to grip the rounded head of the clamp on opposite sides thereof (see FIGS. 6 and 10). A transverse arm can be used to obtain high clamping forces. However, even using an arm, the forces by which the head is clamped in the housing can be insufficient to prevent movement of the drill guide when the jig is in use.

In one aspect the invention provides a drill guide assembly for determining the axis for drilling a bore in a generally dome-shaped bone to receive a component of an orthopaedic joint prosthesis, which comprises: (a) a drill guide sleeve, (b) a carriage in which the drill guide sleeve is mounted towards a first end thereof so that the angular orientation of the drill guide sleeve relative to the carriage can be adjusted about at least one axis, the carriage including at least one threaded angle-adjustment screw which extends between the carriage and the drill guide sleeve by which the angular orientation of the drill guide sleeve can be adjusted, and (c) a platform which can be fastened to the bone, which includes at least three feet depending from the platform to engage the surface of the bone with the bone extending towards the platform into the space between the feet, in which the carriage is mounted relative to the platform so that the drill guide sleeve extends away from the bone, the platform including at least one threaded translation-adjustment screw which extends between the platform and the carriage by which the translational position of the carriage in the plane of the platform, defined by the axis of the translation-adjustment screw, can be adjusted.

The drill guide assembly of the invention has the advantage that the drill guide sleeve and the carriage are held positively against angular and translational adjustment, by the adjustment screws, which act as stays between the sleeve and carriage, and the carriage and platform, respectively. Unwanted movement is prevented subject to frictional forces between the screws and the threaded member on or fastened to the carriage and platform in which the screws are received. Secure locking of the drill guide sleeve is therefore facilitated.

The drill guide assembly of the invention allows the alignment of the drill guide sleeve relative to the bone axis to be finalised after fixing the assembly to the bone. The fixing of the assembly to the bone does not therefore have to be performed accurately because the final alignment of the drill guide sleeve is achieved after fixation, made possible by the translation and angular adjustment of the drill guide sleeve relative to the platform.

The carriage can include only one angle-adjustment screw. The use of only one angle-adjustment screw will restrict the angular orientation of the drill guide sleeve buffeted to the carriage so that it can only be adjusted about one axis. However, a drill guide assembly having only one angle-adjustment screw can be simpler to manufacture than a drill guide assembly having more than one angle-adjustment screw. Therefore the complexity of such a drill guide assembly can be kept to a minimum. Further, the compactness of the drill guide assembly can be maximised. It can be advantageous to maximise the compactness of a drill guide assembly, to ensure that the size of the incision made in the patient is minimised, thereby reducing the healing time and the trauma experienced by the patient.

The platform can include only one translation-adjustment screw. Although a drill guide assembly having only one translation-adjustment screw can facilitate the adjustment of the translation or position of the carriage in the plane of the platform along one axis, the design of such a drill guide assembly can be simpler to manufacture than a drill guide assembly having more than one angle-adjustment screw. Therefore, the complexity of such a drill guide assembly can be kept to a minimum. Further, the compactness of the drill guide assembly can be maximised.

Preferably, the drill guide assembly having only one angle-adjustment screw and only one translation-adjustment screw is configured such that the axis about which the drill guide sleeve rotates and the axis of the translation-adjustment screw are orthogonal relative to each other. This tends to ensure that the adjustment screws extend parallel to each other along the length of the drill guide assembly, thereby maximising the compactness of the drill guide assembly. However, it will be appreciated that the axis about which the drill guide sleeve rotates and the axis of the translation-adjustment screw can be parallel relative to each other, or indeed of any angle relative to each other.

Preferably, the carriage includes at least two angle-adjustment screws. Preferably, the two angle-adjustment screws are configured so that the angular orientation of the drill guide sleeve relative to the carriage can be adjusted about two orthogonal axes. It can be advantageous to provide two orthogonal axes about which the angular orientation of the drill guide sleeve can be adjusted, as it facilitates the adjustment of the angular orientation of the drill guide sleeve relative to the carriage through a solid angle.

Preferably, the angular orientation of the drill guide sleeve can be adjusted about at least one axis, for example two axes, by at least 5° each side of an axis extending perpendicular to the platform. More preferably, the angular orientation of the drill guide sleeve can be adjusted about at least one axis, for example two axes, by at least 10° each side of an axis extending perpendicular to the platform. Especially preferably, the angular orientation of the drill guide sleeve can be adjusted about at least one axis, for example two axes, by at least 15° each side of an axis extending perpendicular to the platform.

Preferably, the angular orientation of the drill guide sleeve can be adjusted about two orthogonal axes.

Preferably, the platform includes at least two translation-adjustment screws. Preferably, the two translation-adjustment screws are configured such that their axes are orthogonal to one another. It can be advantageous to provide two translation-adjustment screws orthogonal to each other, as it facilitates the adjustment of the translational position of the carriage in the plane of the platform in two dimensions rather than one dimension.

Preferably, the translational position of the carriage can be adjusted along at least one axis, for example two axes, in the plane of the platform by at least 0.3 cm. More preferably, the translational position of the carriage can be adjusted along at least one axis, for example two axes, in the plane of the platform by at least 0.5 cm. Especially preferably, the translational position of the carriage can be adjusted along at least one axis, for example two axes, in the plane of the platform by at least 1 cm.

Preferably, the translational position of the carriage can be adjusted along two orthogonal axes in the plane of the platform.

Preferably, the angle-adjustment screw acts on the drill guide sleeve closely adjacent to the point at which the drill guide sleeve is mounted to the carriage. Preferably, the angle-adjustment screw acts on the drill guide sleeve between the point at which the drill guide sleeve is mounted and the end of the drill guide sleeve distil to the bone. However, it will be appreciated that the angle-adjustment screw can act on the drill guide sleeve between the point at which the drill guide sleeve is mounted and the end of the drill guide sleeve proximal to the bone.

It can be advantageous to minimise the distance between the point at which the angle-adjustment screw acts on the drill guide sleeve to the point at which the drill guide sleeve is rotatably mounted within the carriage. This is because the smaller the distance, the smaller the required translational movement of the angle-adjustment screw for any given angle of adjustment. Preferably, the ratio of (a) the distance between the point at which the drill guide sleeve is mounted within the carriage and the end of the drill guide sleeve distal to the bone, to (b) the distance between the point at which the drill guide sleeve is mounted within the carriage and the point at which the angle-adjustment screw acts on the drill guide sleeve, is no less than 2. More preferably, the ratio of (a) to (b) is no less than 3. Especially preferably, the ratio of (a) to (b) is no less than 4.

Preferably, the distance between the point at which the angle-adjustment screw acts on the drill guide sleeve to the point at which the drill guide sleeve is mounted within the carriage is no more than 10 mm. More preferably, the distance between the point at which the angle-adjustment screw acts on the drill guide sleeve to the point at which the drill guide sleeve is mounted within the carriage is no more than 7 mm. Especially preferably, the distance between the point at which the angle-adjustment screw acts on the drill guide sleeve to the point at which the drill guide sleeve is mounted within the carriage is no more than 5 mm.

The drill guide sleeve will generally define a bore in which a drill can be received. The size of the bore should be sufficient for the drill to be sliding fit, with minimum clearance which would allow play to reduce the accuracy of the location of the bore that is drilled in the bone. The sleeve can also be used to locate surgical instruments other than drill bits. Suitable drill guide sleeves might have bores with an internal transfers dimension of, for example, 2 to 5 mm.

Preferably, the length of the drill guide sleeve is no less than 1 cm. More preferably, the length of the drill guide sleeve is no less than 2 cm. Especially preferably, the length of the drill guide sleeve is no less than 3 cm. Preferably, the length of the drill guide sleeve is no more than 6 cm. More preferably, the length of the drill guide sleeve is no more than 5 cm. Especially preferably, the length of the drill guide sleeve is no more than 4 cm. Preferably, the ratio of (i) the total length of the drill guide sleeve, to (ii) the distance between the end of the drill guide sleeve distal to the bone and the point at which it is mounted within the carriage, is less than 4. More preferably, the ratio of (i) to (ii) is less than 3. Especially preferably, the ratio of (i) to (ii) is less than 2. Most preferably, the drill guide sleeve is mounted at its first end (i.e. the ratio of (i) to (ii) is 1).

It can be advantageous that the distance between the point about which the drill guide sleeve is mounted within the carriage, and the surface of the bone, is minimised. This reduces the translational distance through which the axis, defined by the length of the drill guide sleeve, moves across the surface of the bone for any given angle of rotation of the drill guide sleeve relative to the carriage. This is a known problem of drill guide assemblies in which the translational position of the drill guide sleeve is positioned accordingly, and then the angular orientation of the drill guide sleeve is adjusted, causing the point at which the axis of the drill guide sleeve meets the bone (i.e. the point at which the drill or other instrument would contact the bone) to move, therefore requiring the translational position of the drill guide sleeve to be adjusted again, and so on. Therefore, it can be advantageous that the feet of the platform are configured so that the generally dome shaped surface of the bone extends towards the platform in the space between the feet, to minimise the distance between the point about which the drill guide sleeve is mounted within the carriage and the surface of the bone.

Preferably, the drill guide sleeve is mounted in the carriage so that the axis around which the angular orientation of the drill guide sleeve is adjusted is closely adjacent to the plane of the platform defined by the axis of the translation-adjustment screw. More preferably, the drill guide sleeve is mounted in the carriage so that the axis around which the angular orientation of the drill guide sleeve is adjusted lies in the plane of the platform defined by the axis of the translation-adjustment screw.

Preferably, the platform includes three feet depending from the platform which can engage the surface of the bone. More preferably, the platform includes four feet depended from the platform which can engage the surface of the bone. Preferably, the feet are elongate members tapered towards a sharp tip so that the feet easily engage the surface of the bone.

Preferably, the feet have a generally triangular cross-section. However, it will be appreciated that any member capable of engaging the surface of the bone to prevent movement of the drill guide assembly can be used. For example, the feet might be conical, or pyramidal.

Preferably, the distance between the tip of the feet and the base of the platform from which the feet depend is not more than 1 cm. More preferably, the distance between the tip of the feet for engaging the surface of the bone, and the base of the platform from which the feet depend is not more than 0.5 cm. Especially preferably, the distance between the edge of the feet for engaging the surface of the bone, and the base of the platform from which the feet depend is not more than 0.2 cm.

Preferably, the distance between the two closest feet is no less than 2 cm. More preferably, the distance between the two closest feet is no less than 1.5 cm. Especially preferably, the distance between the two closest feet is no less than 1 cm.

Preferably, the ratio of the distance between the two closest feet to the distance between the tip of the feet and the base of the platform from which the feet depend is no less than 4. More preferably, the ratio of the distance between the two closest feet to the distance between the tip of the feet and the base of the platform from which the feet depend is no less than 3. Especially preferably, the ratio of the distance between the two closest feet to the distance between the tip of the feet and the base of the platform from which the feet depend is no less than 2.

Preferably, the platform comprises means for fixing the drill guide assembly to the bone. Preferably, the platform comprises at least one bore for receiving a fastener by which the drill guide assembly can be fixed at the bone. Preferably, the fastener is a pin which can be easily hammered into the bone. However, it will be appreciated that other types of fastener can be used, for example, a bone screw. More preferably, the platform has two or more holes for receiving a fastener. Preferably, the axis defined by the length of the bore is not perpendicular to the plane of the platform. It has been found that when bores are angled so that they are not perpendicular to the plane of the platform, the force required to remove the platform from the bone when it has been fastened is significantly increased. Preferably, two bores are provided on opposing sides of the platform, who's axes defined by their length, cross at some point on the side of the platform proximal to the bone.

Preferably, the platform defines a region within which the carriage fits. Preferably, the platform comprises a mechanism which facilitates the translational movement of the carriage within the region. Preferably, the carriage comprises a mechanism which facilitates the translational movement of itself within the region of the platform. Preferably the sides of the platform defining the region comprise ledges which extend into the region, and the carriage comprises corresponding recesses, so that the carriage can sit on the ledges. The co-efficient of friction between the ledges of the platform and the recesses of the carriage must be sufficiently small to allow the carriage to slide easily along the ledges. However, it will be appreciated that any mechanism facilitating the translational movement of the carriage within the platform can be used.

The drill guide assembly can include a nut connected to the drill guide sleeve, wherein the angle-adjustment screw extends through the nut, the nut having a thread which mates with the thread on the screw. Therefore, when the angle-adjustment screw is rotated about its axis, the angle-adjustment screw moves through the nut. The angle-adjustment screw can be fastened at a second end to the carriage so that translational movement of the angle-adjustment screw relative to the carriage is inhibited. Therefore, when the angle-adjustment screws moves through the nut connected to the drill guide sleeve, the drill guide sleeve is caused to rotate about its mounting in the carriage.

Preferably, drill guide assembly includes a nut connected to the carriage, wherein the angle-adjustment screw extends through the nut, the nut having a thread which mates with the thread on the screw. Therefore, when the angle-adjustment screw is rotated about its axis the angle-adjustment screw moves through the nut. Preferably, the nut is rotatably connected to the carriage so that the nut is capable of rotating about an axis which passes through and is perpendicular to the axis of the angle-adjustment screw extending through the nut. However, it will be appreciated that the nut need not be rotatably connected to the carriage.

Preferably, the angle-adjustment screw is fastened at a first end to the drill guide sleeve so that translational movement of the angle-adjustment screw relative to the drill guide sleeve is inhibited. Therefore, when the angle-adjustment screw moves through the nut connected to the carriage, the drill guide sleeve moves with the angle-adjustment screw, thereby causing the drill guide sleeve to rotate about its mounting in the carriage.

Preferably, the angle-adjustment screw is fastened to the drill guide sleeve so that the angle-adjustment screw is pivotable about the point at which it is fastened to the sleeve, around an axis which extends through and perpendicularly to the axis of the angle-adjustment screw. Preferably, the fastening by which the angle-adjustment screw is fastened to the drill guide sleeve comprises a retaining shaft having a smooth bore which receives a smooth end of the angle-adjustment screw so that the screw is rotatable within the shaft. Preferably, the drill guide sleeve comprises at least one arm extending away from the drill guide sleeve, to which the shaft is connected. Especially preferably, the drill guide sleeve comprises two arms extending away from the drill guide sleeve, to which the shaft is connected. Preferably, the at least one arm of the drill guide sleeve extends perpendicularly to the length of the drill guide sleeve. However, it will be appreciated that the arm or arms do not necessarily need to extend perpendicular to the drill guide sleeve. Further, it will be appreciated that the angle-adjustment screw can be retained by the drill guide sleeve by any means which allow the screw to rotate within, and pivot about said means.

It will be appreciated that the angle-adjustment screw need not be fastened to the drill guide sleeve so that the angle-adjustment screw is pivotable about the point at which it is fastened to the sleeve, around an axis which extends through and perpendicularly to the axis of the angle-adjustment screw. For example, the angle-adjustment screw can be fastened to a sheath which is capable of sliding along the length of the drill guide sleeve.

Preferably, the carriage includes at least one arm extending away from the platform, to which the nut retaining the angle-adjustment screw within the carriage is connected. More preferably, the carriage includes two arms extending away from the platform, to which the nut retaining the angle-adjustment screw within the carriage is connected, and extends between the arms. However, it will be appreciated that the nut within which the angle-adjustment screw is retained within the carriage need not necessarily be retained within an arm or arms extending away from the carriage.

Preferably, the drill guide assembly includes a nut connected to the platform, wherein the translation-adjustment screw extends through the nut, the nut having a thread which mates with the screw. Preferably, movement between the nut and platform is inhibited. Preferably, the translation-adjustment screw is fastened at a first end to the carriage so that the translational movement of the translation-adjustment screw relative to the carriage is inhibited. Therefore, as the translation-adjustment screw moves, the carriage moves in the same direction by an equal distance. It will be appreciated that a nut can be connected to the carriage, wherein the translation-adjustment screw extends through the nut, the nut having a thread which mates with the screw. Further, the translation-adjustment screw can be fastened to the platform so that translational movement of the translation-adjustment screw relative to the platform is inhibited, therefore rotation of the translation-adjustment screw causes the carriage to travel along the translation-adjustment screw.

Preferably, the depth of the thread of the adjustment screws is no less than 0.2 mm. More preferably, the depth of the thread of the adjustment screws is no less than 0.5 mm. Especially preferably, the depth of the thread of the adjustment screws is no less than 0.7 mm.

Preferably, the depth of the thread of the nut through which an adjustment screw extends is no less than 0.2 mm. More preferably, the depth of the thread of the nut through which an adjustment screw extends is no less than 0.5 mm. Especially preferably, the depth of the thread of the nut through which an adjustment screw extends is retained is no less than 0.7 mm.

Preferably, the pitch of the thread of the adjustment screws is no more than 1 mm. More preferably, the pitch of the thread of the adjustment screws is no more than 0.5 mm. Especially preferably, the pitch of the thread of the adjustment screws is no more than 0.3 mm.

Preferably, the assembly includes an alignment stylus connected to the drill guide sleeve to move with the drill guide sleeve relative to the platform, the stylus including a first length which extends in a direction generally towards the bone, to facilitate the assessment of the alignment of the drill guide sleeve relative to anatomical features of the bone. Preferably, the stylus can be moved rotatably around the drill guide sleeve. Preferably, the stylus can be moved relative to the platform independently of any movement of the drill guide sleeve relative to the platform.

The stylus can include a second limb extending from the first limb in a direction generally towards the axis of the assembly, the second limb having a stylus tip. The second limb can be used to determine the position of the drill guide sleeve relative to the surface of the longitudinally extending side wall of a bone where the frame is attached at one end thereof. For example, when the frame is attached to the spherical head part of a long bone such as a femur, approximately on the axis of the femoral neck, the second limb of the stylus can trace around the femoral neck or the base of the spherical head, to assess the transactional alignment of the drill guide sleeve, or its angular alignment or both.

Preferably, the length of at least one of the first and second limbs of the stylus is adjustable. For example, one or each of the limbs can comprise first and second telescoping parts. The length of the or each adjustable limb should be capable of being locked, for example by means of a threaded fastener.

The instrument will generally be made from metallic materials which are conventionally used in the manufacture of surgical instruments. Certain stainless steels are particularly preferred.

Embodiments of the present invention will now be described by way of example only with reference to the accompanying drawings, in which:

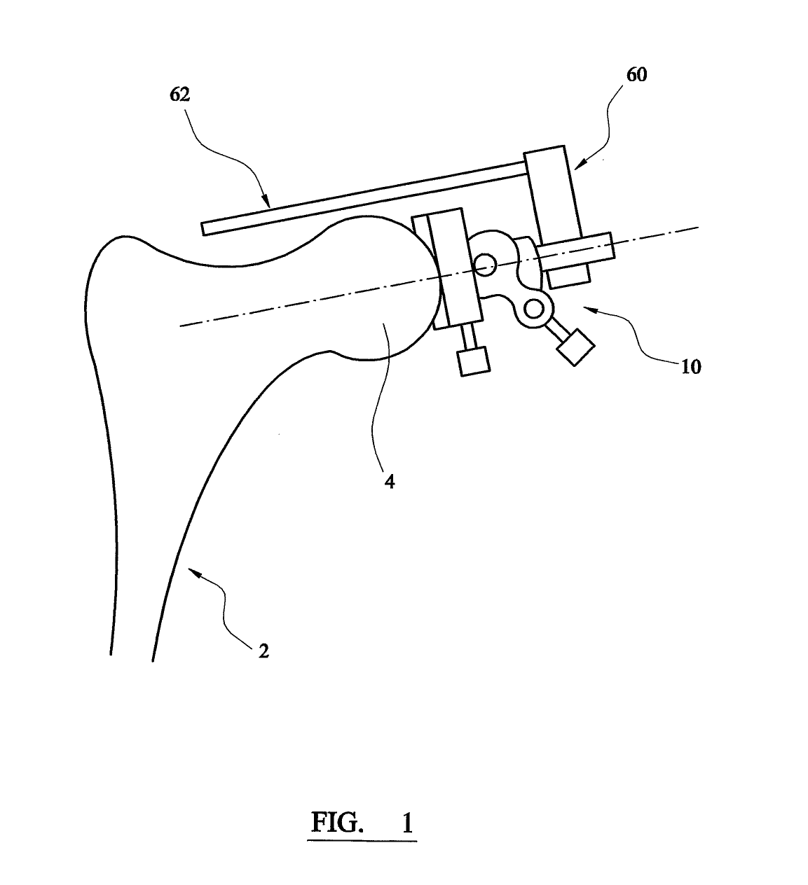

FIG. 1 is a side view of an alignment stylus connected to the drill guide assembly according to the invention, mounted on the femur head.

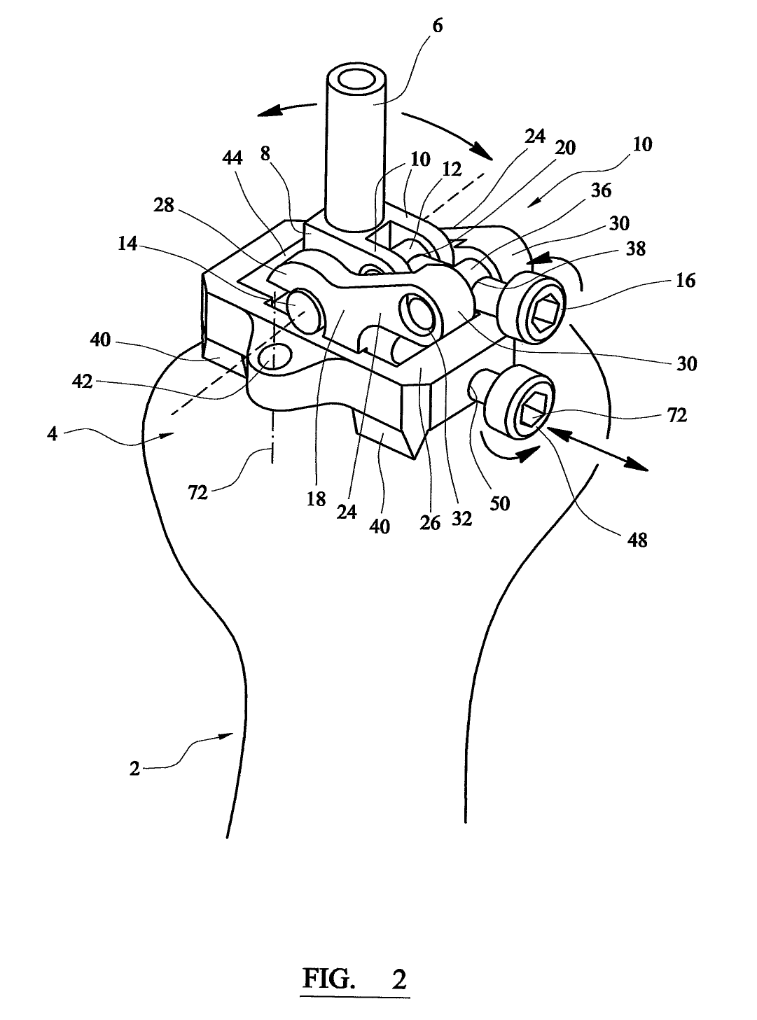

FIG. 2 is an isometric view of the drill guide assembly shown in FIG. 1, mounted on the femur head.

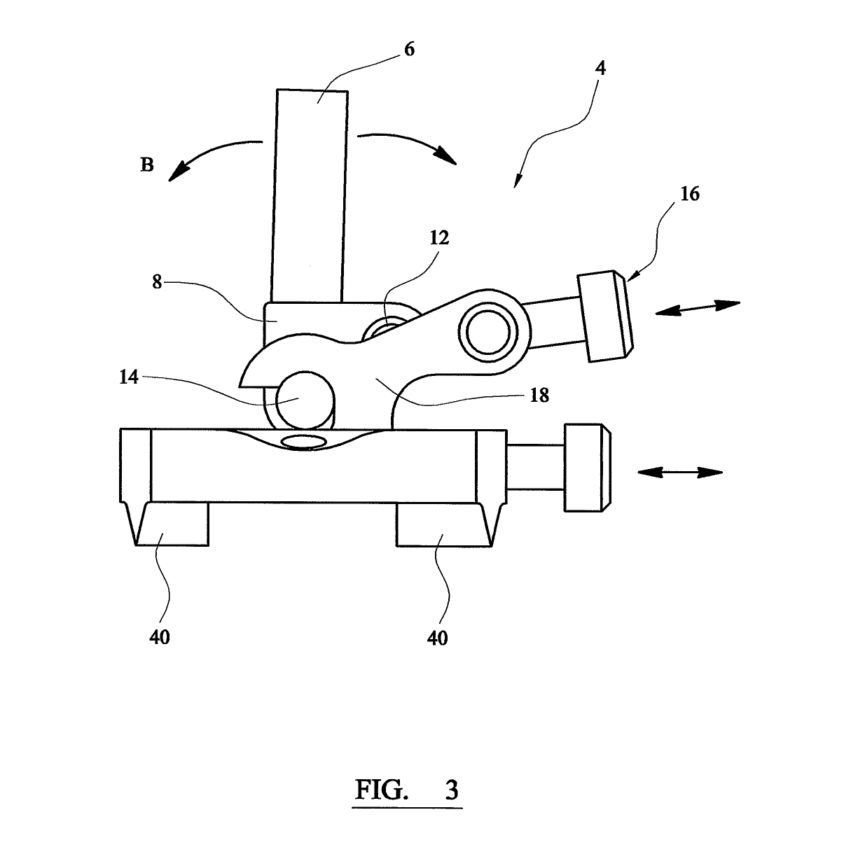

FIG. 3 is a side view of the drill guide assembly shown in FIG. 1.

FIG. 4 is an end view of the drill guide assembly shown in FIG. 1 mounted on the femur head.

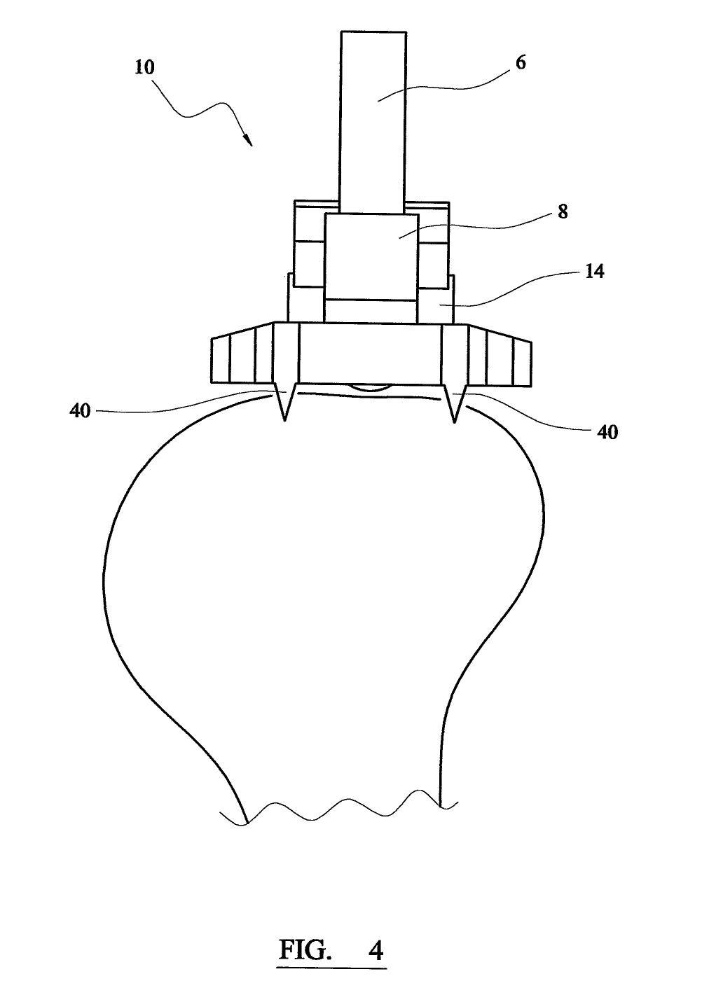

FIG. 5 is a bottom view of the drill guide assembly shown in FIG. 1.

FIG. 6 is a top view of the drill guide assembly shown in FIG. 1.

Referring to the drawings, FIG. 1 shows the upper portion of a femur 2, including the head 4 which is received in the patient's acetabulum during articulation of the joint, and a drill guide assembly 10 according to the present invention fastened to the femur head. The femur is shown in isolation to show how the present invention can be put into effect to prepare the femur for implementation of a “resurfacing” joint prosthesis component. This can be used to provide a hard-wearing bearing surface on the head of the femur, which can articulate with an appropriate prosthesis component which is implanted in the acetabulum. The use of such surgical techniques has the advantage that most of the femoral tissue is preserved.

The preparation of the head of the femur to receive a resurfacing prosthesis component involves the formation of a bore along the axis of the head, to receive the stem of the component. The external surface of the head is prepared by the formation of two rotationally symmetrical reamed surfaces. A first surface is aligned parallel or at a predetermined angle relative to the axis of the head. A second surface extends between the first surface and the axis, the angle between the first and second surfaces being about 135°.

The preparation of the external surface of the head requires determination of the axis for the component.

FIG. 2 shows the drill guide assembly 10 in more detail. The drill guide assembly generally comprises a drill guide sleeve 6, rotatably mounted within a carriage 18, which is slidably mounted within a platform 26. The drill guide further comprises angular 16 and translation 48 adjustment screws for adjusting the angular orientation of the drill guide sleeve 6 relative to the carriage 18, and for adjusting the translational position of the carriage within the platform 26, respectively.

Translation-adjustment screw 48 (shown best in FIG. 5) has a cylindrical head portion 64, threaded shaft portion 66 adjacent the head portion, smooth shaft portion 68 adjacent the threaded shaft portion, and cylindrical stopper portion 70 adjacent the smooth shaft portion. Head part 64 comprises a hexagonal shaped recess 72 for receiving a similar shaped tool (not shown) for imparting a rotational force on the screw. The diameter of smooth shaft part 68 is smaller than that of the threaded shaft part 66. The diameter of the cylindrical stopper part 70 is generally equal to that of the threaded shaft part 66. The diameter of head part 64 is larger than that of the threaded shaft part 66. Angle-adjustment screw 16 has an identical configuration to that of the translation-adjustment screw 48.

The bore along the length of the drill guide sleeve 6 is slightly bigger than the size of a drill (not shown) which has to extend through the drill guide sleeve to create a bore in the patient's bone. The drill guide sleeve 6 has two co-axial cylindrical projections 14 extending perpendicularly away from the drill guide sleeve, at a first end thereof proximal to the bone. The cylindrical projections 14 are rotatably mounted within a bearing provided by the carriage and the platform, thereby facilitating angular adjustment of the drill guide sleeve within the carriage, as described in more detail below.

The drill guide sleeve 6 also includes a structure 8 for receiving the angle-adjustment screw 16. The structure includes two parallel arms 10 extending perpendicularly away from the drill guide sleeve 6, which house a retaining shaft 12 towards their ends distil to the drill guide sleeve. The retaining shaft 12 extends between the arms 10 and is rotatable about its axis. The retaining shaft 12 has a cylindrical bore 20 extending transversely through the portion of the shaft between the arms 10. The surface of the bore 20 is smooth, and is capable of receiving the smooth shaft portion of the angle-adjustment screw 16 so that the angle-adjustment screw can easily rotate within the bore 20. The diameter of the bore 20 is smaller than that of the stopper portion 70 and threaded shaft portion 66, thereby inhibiting translational movement of the angle-adjustment screw 16 through the retaining shaft 12.

The carriage 18 of the drill guide assembly 10 comprises a hollow square base part 22 slidably mounted within the platform 16. The carriage 18 further comprises two arms 24 extending away from the platform 16, on opposing sides of the base 22 of the carriage. The first ends 28 of the arms 24 proximal to the base part 22 of the carriage 18 contain a bearing surface for the cylindrical projections 14 of the drill guide sleeve 6, so that the projections 14 are rotatably retained within the drill guide assembly between the arms 24 of the carriage 18 and the platform 26. The second ends 30 of the arms 24 distil to the base part 22 comprise smooth cylindrical bores 32. The bores 32 at the second end 30 of the arms 24 provide a bearing surface for a shaft 36 which extends between the second ends 30, and is rotatable within the bores along its axis. The shaft 36 includes a bore 36 extending transversely through it and is capable of receiving the threaded part of the angle-adjustment screw 16. The bore 38 of the shaft 36 extending between the second ends 30 of the arms 24 of the carriage 18 has a thread which mates with the thread on the angle-adjustment screw 16, so that when the screw is rotated, the screw travels through the shaft.

The platform 26 comprises a hollow rectangular body part within which the carriage 18 is slidably retained, and four feet 40 depending from the body for engaging the surface of the femoral head 4. The feet 40 are elongate members tapered towards a sharp tip. The feet 40 and the body of the platform 26 are one piece. The platform 26 further comprises bores 42 for receiving pins or screws (not shown) for securing the platform 26 to the femoral head floor. The bores 42 are inwardly angled so that the axes 72 defined by the length of the bores would cross at a point on the side of the platform 26 distal to the drill guide sleeve 6.

The inside surface 44 of the hollow rectangular body part 26 defines a region within which the carriage 18 is retained. Ledges 46 are provided on two opposing sides of the internal surface 44 upon which corresponding recesses in the base part 22 of the carriage 18 sits, so that the carriage is capable of sliding along the ledges. The translation-adjustment screw 48 extends through a bore 50 in one of the sides of the platform perpendicular to the ledged sides of the platform. The bore 50 has a thread which mates with the thread on the translation-adjustment screw 48 so that when the screw is rotated, the screw travels through the bore. The smooth part of the translation-adjustment screw 48 is retained within a smooth bore 52 extending through a side of the body 22 of the carriage 18, so that it is rotatable within the bore. The diameter of the smooth bore 52 of the carriage 18 is smaller than that of the stopper portion 70 and threaded shaft portion 66 so that translational movement of the translation-adjustment screw 48 through the smooth bore is inhibited.

The assembly of the invention can be used to locate the axis on which the spherical head of a femur or similar long bone is prepared for a section prior to implementation of a joint prosthesis component. In a first step, the axis of the bone is determined approximately, for example by eye, using instruments, with a reference to other anatomical features of the patient's bone. Instruments which can be used for this purpose in relation to the patient's femur are disclosed in WO-A-03/026517 and WO-A-03/026518. The platform 26 is then attached relative to this axis by means of fasteners which pass through the bores 42, into the bone tissue.

As shown in FIG. 1, the assembly of the invention includes a stylus 60 which can be rotated about the axis which is defined by the drill guide sleeve 6. The stylus comprises a first limb 62 which extends in a direction parallel to the drill guide sleeve 6, away from the drill guide sleeve towards the bone. The stylus can also include a second limb (not shown) extending from the first limb in a direction generally towards the axis of the drill guide sleeve 6, the second limb having a stylus tip.

The stylus can be rotated about the axis of the bone to determine the position of the drill guide relative to the surface of the bone which can be traced out using the stylus. When the stylus can be rotated about the spherical head of the bone with the constant distance between the stylus tip and the bone surface, the drill guide will be aligned with the axis of the head. This will generally be the desired location for drilling the bone. However, in some applications, it can be desired for the drill guide to be located off the bone axis.

1. A drill guide assembly for determining the axis for drilling a bore in a generally dome-shaped bone to receive a component of an orthopaedic joint prosthesis, comprising:
a. a drill guide sleeve;
b. a carriage in which the drill guide sleeve is mounted towards a first end thereof so that the angular orientation of the drill guide sleeve relative to the carriage can be adjusted about at least one axis, the carriage including at least one threaded angle-adjustment screw which extends between the carriage and the drill guide sleeve by which the angular orientation of the drill guide sleeve can be adjusted; and
c. a platform which can be fastened to the bone, which includes at least three feet depending from the platform to engage the surface of the bone with the bone extending towards the platform into the space between the feet, in which the carriage is mounted relative to the platform so that the drill guide sleeve extends away from the bone, the platform including at least one threaded translation adjustment screw which extends between the platform and the carriage by which the translational position of the carriage in the plane of the platform, defined by the axis of the translation-adjustment screw, can be adjusted.

2. The drill guide assembly of claim 1, wherein the carriage includes only one angle-adjustment screw.

3. The drill guide assembly of claim 1, wherein the platform includes only one translation-adjustment screw.

4. The drill guide assembly of claim 2, wherein the axis about which the drill guide sleeve rotates and the axis of the translation-adjustment screw are orthogonal relative to each other.

5. The drill guide assembly of claim 1, wherein the carriage includes two angle-adjustment screws.

6. The drill guide assembly of claim 1, wherein the platform includes two translation-adjustment screws.

7. The drill guide assembly of claim 5 wherein the adjustment screws are arranged such that their axes are orthogonal relative to each other.

8. The drill guide assembly of claim 1, wherein the angle-adjustment screw acts on the drill guide sleeve closely adjacent to the point at which the drill guide sleeve is mounted in the carriage.

9. The drill guide assembly, of claim 1, further comprising a nut connected to the carriage, wherein the angle-adjustment screw extends through the nut, the nut having a thread which mates with the thread on the screw.

10. The drill guide assembly of claim 9, wherein the angle-adjustment screw is fastened at a first end to the drill guide sleeve so that translational movement of the angle-adjustment screw relative to the drill guide sleeve is inhibited.

11. The drill guide assembly of claim 9, wherein the nut is rotatably connected to the carriage so that the nut is capable of rotating about an axis which passes through and is perpendicular to the axis of the angle-adjustment screw extending through the nut.

12. The drill guide assembly of claim 1, further comprising a nut connected to the platform, wherein the translation-adjustment screw extends through the nut, the nut having a thread which mates with the screw.

13. The drill guide assembly of claim 12, wherein the translation-adjustment screw is fastened at a first end to the carriage so that the translational movement of the translation-adjustment screw relative to the carriage is inhibited.

14. The drill guide assembly of claim 12, wherein movement between the nut and the platform is inhibited.

15. The drill guide assembly of claim 1, further comprising an alignment stylus connected to the drill guide to move with the drill guide relative to platform, the stylus including a first limb which is directed towards the bone to facilitate assessment of the alignment of the drill guide sleeve relative to anatomical features of the bone.

16. The drill guide assembly of claim 15, wherein the stylus can be moved rotatably around the drill guide sleeve.

17. The drill guide assembly of claim 15, wherein the stylus includes a second limb extending from the first limb in a direction generally towards the axis of the drill guide sleeve.

18. The drill guide assembly of claim 17, wherein the length of at least one of the first and second limbs of the stylus is adjustable.
